(19) United States
(12) Patent Application Publication
(10) Pub. No.: US 2004/0006343 A1
(43) Pub. Date: Jan. 8, 2004

(54) Auxiliary vertebrae connecting device

(76) Inventor: Lionel C. Sevrain, West Palm Beach, FL (US)

Correspondence Address: Mr. Tom Hamill, National Patent Services, Suite 308, 2101 Crystal Plaza Arcade, Arlington, VA, 22202, US

(21) Appl. No.: 10/449,702

(22) Filed: Jun. 2, 2003

Related U.S. Application Data
(63) Continuation-in-part of application No. 10/358,398, filed on Feb. 5, 2003.
Application No. 10/358,398 is a continuation-in-part of application No. PCT/CA01/00739, filed on May 25, 2001.
(60) Provisional application No. 60/206,810, filed on May 25, 2000.
(60) Provisional application No. 60/264,309, filed on Jan. 29, 2001.

(51) Int. Cl. A61B 17/56

(52) U.S. Cl. 606/61

(57) ABSTRACT

A connecting device for use in attaching together at least two adjacent vertebrae comprises upper and lower plate members adapted to be fixedly secured respectively to adjacent upper and lower vertebrae via fasteners, e.g. screws. The upper and lower plate members are provided with respective male and female guide elements that are slidable engaged together so as to allow a relative translational displacement between the upper and lower plate members in response to a similar relative displacement between the upper and lower vertebrae that alters a spacing therebetween. The male guide element comprises longitudinal guide ways, and the female guide element comprises a channel for slidably receiving the male guide element and a pair of side guides extending inwardly and slidably engaged in the guide ways of said male guide element. The female guide element defines a longitudinal slot, and the male guide element comprises a pin extending perpendicularly to a direction of the aforementioned relative displacement and engaged in the slot. A method includes: removing at least part of a disc from an intervertebral space defined between a pair of adjacent vertebrae that include upper and lower vertebrae; and securing first and second plate members respectively to the upper and lower vertebrae, said first and second plate members being capable of relatively displacing for accommodating a change in a distance between the upper and lower vertebrae; wherein a bone graft is positioned in the intervertebral space.

### CROSS-REFERENCE

This Application is a Continuation-in-Part of U.S. application Ser. No. 10/358,398 filed on Feb. 5, 2003, itself a Continuation-in-Part of the U.S. National Entry Application (Ser. No. not yet known), filed on Nov. 25, 2002, of PCT/CA01/00739 filed on May 25, 2001 designating the United States and claiming priority on U.S. Provisional Applications Serial No. 60/206,810 filed on May 25, 2000 and No. 60/264,309 filed on Jan. 29, 2001.

### BACKGROUND OF THE INVENTION

(a) Field of the Invention

(b) Description of prior art

When a cervical disc is anteriorly removed (e.g. using the Smith-Robinson surgical technique) from between two adjacent vertebrae, for instance to liberate roots and/or spinal cord which are compressed by a degenerated disc or to remove a damaged disc, it is known to fuse both these vertebrae together (i.e. osteosynthesis by way of an anterior cervical plate) to provide stability to the rachis although this results in loss of mobility and damping. This rigidification (on one or more successive discs) induces greater stresses to the natural discs located adjacently above and below the removed disc(s) thereby causing a premature ageing of these natural discs, and also creates experimental conditions for the formation of discal hernias on the adjacent levels.

U.S. Pat. No. 5,258,031 issued to Salib et al. discloses a non-compressible prosthetic disc of the ball-and-socket type where the male and female members are fixed to respective upper and lower plates that are secured to adjacent upper and lower vertebrae by way of screws. The disc prosthesis is thus adapted to replace a natural disc of the lumbar spine and it provides six degrees of freedom such as to substantially reproduce the normal intervertebral pivoting movements, except compression.

U.S. Pat. No. 5,865,846 and No. 6,001,130 both issued to Bryan et al. respectively on Feb. 2 and Dec. 14, 1999 are similar (although the latter is more detailed) in each teaching a disc prosthesis comprising a resilient body, of varying stiffness from a substantially stiff exterior annular gasket to a relatively supple central nucleus. The disc prosthesis is adapted to be installed in the intervertebral space with concave-convex elements at least partly surrounding the resilient body to retain it in the intervertebral space. These elements include a pair of L-shaped supports mounted to respective adjacent upper and lower vertebrae with screws that extend through the vertical leg or wing sections of the L-shaped supports. The horizontal leg or wing sections of the L-shaped supports extend in the intervertebral space and surround the resilient body on opposite upper and lower sides thereof. It is possible for the vertical and horizontal leg sections of each L-shaped support to be hinged together at the anterior faces of the vertebrae but only for adjustment during installation of the prosthesis and not to act as a hinge after installation. There may be two or more disc prostheses disposed in series between three or more adjacent vertebrae.

U.S. Pat. No. 5,755,796 issued on May 26, 1998 to Ibo et al. discloses a cervical intervertebral disc prosthesis also of the ball-and-socket type which allows for a pivotal movement between two adjacent vertebrae.

U.S. Pat. No. 5,556,431 issued on Sep. 10, 1996 to Biittner-Janz also teaches a disc prosthesis somewhat similar in function to that of aforementioned U.S. Pat. No 6,001,130, although its two anchoring plates do not extend along the anterior faces of the vertebrae The screws holding the anchoring plates to the vertebrae are engaged in the vertebrae from the intervertebral face thereof The prosthesis core has a peripheral rim to limit its range of movements. Anchoring teeth are provided on the plates for penetrating, under load, the vertebrae.

U.S Pat. No. 3,426,364 issued on Feb. 11, 1969 to Lumb describes a spinal prosthesis to replace natural vertebrae which had to be removed A spring member may extend in the prosthesis.

U.S. Pat. No. 5,562,738 issued to Boyd et al. on Oct. 8, 1996 is similar to above-described U.S. Pat. No. 5,258,031.

U.S. Pat. No. 5,171,280 issued on Dec. 15, 1992 to Baumgartner discloses an inter-vertebral prosthesis which includes a coiler body able to rotate onto a fixed base with a flexible elastic hollow body extending from the coiler body and adapted to receive therein a filling medium through a valve, The prosthesis once implanted and filled with an incompressible medium, is able to absorb radial forces exerted upon the periphery via the incompressible medium in the elastic hollow body. The prosthesis can be inserted in the inter-vertebral region through a small opening.

U.S. Pat. No. 3,875,595 issued on Apr. 8, 1975 to Froning discloses an intervertebral disc prosthesis in the form of a collapsible plastic bladder-like member that has the shape of the nucleus pulposis of a natural inter-vertebral disc. After removal of the degenerated natural nucleus pulposis, the prosthesis, in its collapsed position, is inserted through a stem and into the inter-somatic space, and a filling medium is then inserted through the stem and into the prosthesis to inflate it to a natural form. The stem is then severed just upstream of a valve thereof such that the valve remains implanted with the prosthesis.

U.S. Pat. No. 4,772,287 and No. 4,904,260 which issued respectively on Sep. 20, 1998 and Feb. 27, 1990 both in the names of Ray et al. describe the implantation of two prosthetic disc capsules side-by-side into a damaged disc of a human spine.

U.S. Pat. No. 6,022,376 issued on Feb. 8, 2000 to Assell et al. discloses a capsule-shaped prosthetic spinal disc nucleus for implantation into a human intradiscal space, made of a substantially inelastic constraining jacket surrounding an amorphous polymer core with the constraining jacket having a fixed maximum volume and defining a height, while the amorphous polymer core fills an initial volume of the constraining jacket and develops an internal pressure.

U.S. Pat. No. 5,192,326 and No. 5,047,055 which issued respectively on Mar. 9, 1993 and Sep. 10, 1991 both in the name of Bao et al. teach a prosthetic nucleus adapted to be implanted in the intersomatic space of a spine and which is formed of a multiplicity of hydrogel beads which are covered by a semi-permeable membrane. This prosthetic nucleus is adapted to conform, when hydrated, to the general shape of the natural nucleus. The prosthetic nucleus is surrounded by the natural annulus fibrous Vertebral end plates cover the superior and inferior faces of the prosthetic nucleus.

U.S. Pat. No. 4,863,477 issued on Sep. 5, 1989 to Monson discloses a synthetic inter-vertebral disc prosthesis which is made of two halves which, after having been joined together, are implanted in the intersomatic space in place of a removed natural disc A fluid, such as a saline solution, is then injected into the interior cavity of the prosthesis to provide the required amount of resiliency in the disc prosthesis thereby restoring proper vertebral spacing and facilitating flexibility of the spine.

U.S. Pat. No. 5,976,186 issued on Nov. 2, 1999 to Bao et al. discloses a hydrogel inter-vertebral disc nucleus adapted to be inserted in the inter-somatic space through an opening in the natural annulus for replacing the natural nucleus. The hydrogel disc is adapted to essentially fill the intervertebral nuclear disc cavity upon absorbing sufficient water from the body fluids.

Furthermore, when a disc, or part thereof, is removed from between two adjacent vertebrae, there is often installed a filling, typically a bone graft, in the intersomatic space located between the two vertebrae. A plate is used to fixedly connect both vertebrae, via screws that extend though the plate and into both vertebrae. The bone graft takes months to fuse with these two vertebrae and, while doing so, the bone graft reduces in size (graft subsidence), i.e. contracts, e.g. from 1 to 2 mm for a bone graft having an initial height of 8 to 10 mm. This causes great stress on the rigid unit formed by the two vertebrae and by the plate and screws that connect these two vertebrae together, thereby resulting in significant problems, e.g. the screws can break, the plate can fall, and generally there is breakage of the screws at plate-screw interface.

### SUMMARY OF THE INVENTION

It is therefore an aim of the present invention to provide a novel device for connecting two or more adjacent vertebrae of the human rachis.

Therefore, in accordance with the present invention, there is provided a connecting device for use in attaching together at least two adjacent vertebrae, comprising first and second plate members adapted to be fixedly secured respectively to upper and lower vertebrae of a pair of adjacent vertebrae, said first and second plate members being provided with respective first and second mating members, said first and second mating members being engaged so as to allow a relative displacement between said first and second plate members in response to a similar relative displacement between the upper and lower vertebrae that alters a spacing there between.

Also in accordance with the present invention, there is provided a method of connection of at least two adjacent vertebrae, comprising the steps of:

a) removing at least part of a disc from an intervertebral space defined between a pair of adjacent vertebrae that include upper and lower vertebrae;

b) securing first and second plate members respectively to the upper and lower vertebrae, said first and second plate members being capable of relatively displacing for accommodating a change in a distance between the upper and lower vertebrae;

wherein after step a), a bone graft is positioned in the intervertebral space.

### BRIEF DESCRIPTION OF THE DRAWINGS

Having thus generally described the nature of the invention, reference will now be made to the accompanying drawings, showing by way of illustration a preferred embodiment thereof, and in which.

FIG. 1 is a schematic side elevational view of a disc prosthesis in accordance with a first embodiment of the present invention and shown mounted to a pair of adjacent vertebrae;

FIG. 1A a schematic perspective view of the disc prosthesis of FIG. 1;

FIGS. 1B and 1C are schematic front and side elevational views of the disc prosthesis of FIG. 1A shown in an extended position thereof between the adjacent vertebrae;

FIGS. 1D and 1E are schematic front and side elevational views of the disc prosthesis of FIG. 1A shown in a flexed position thereof between the adjacent vertebrae;

FIG. 2 is a schematic side elevational view of a disc prosthesis in accordance with a second embodiment of the present invention and shown mounted to a pair of adjacent vertebrae;

FIG. 2A is a schematic perspective view of the disc prosthesis of FIG. 2;

FIGS. 2B and 2C are schematic front and side elevational views of the disc prosthesis of FIG. 2A shown in an extended position thereof between the adjacent vertebrae;

FIGS. 2D and 2E are schematic front and side clevational views of the disc prosthesis of FIG. 2A shown in a flexed position thereof between the adjacent vertebrae;

FIG. 3 is a schematic front elevational view of the disc prosthesis of FIG. 2;

FIG. 4 is a schematic perspective view of a disc prosthesis in accordance with a third embodiment of the present invention;

FIGS. 5 and 6 are respectively front perspective and side elevational views of the disc prosthesis of FIG. 4, shown mounted to a pair of adjacent vertebrae;

FIG. 7 is a perspective view of a device also in accordance with the present invention for connecting two adjacent of adjacent vertebrae;

FIGS. 8A and 8B are front and side elevational views of the device of FIG. 7 shown mounted to two adjacent vertebrae and in an extended position thereof; and

FIGS. 9 and 10 are cross-sectional views taken respectively along lines 9-9 and 10-10 of FIG. 7.

### DESCRIPTION OF THE PREFERRED EMBODIMENTS

As discussed hereinabove, when a cervical disc is anteriorly removed (e.g. using the Smith-Robinson surgical technique) from between two adjacent vertebrae, for instance to liberate roots and/or spinal cord which are compressed by a degenerated disc or to remove a damages disc, it is known to fuse both these vertebrae together (i.e. osteosynthesis by way of an anterior cervical plate) to provide stability to the rachis although this results in loss of mobility and damping This rigidification (on one or more successive discs) induces greater stresses to the natural discs located adjacently above and below the removed disc(s) thereby causing a premature ageing of these natural discs, and also creates experimental conditions for the formation of discal hernias on the adjacent levels.

To overcome at least in part these disadvantages, the present invention proposes a new disc prosthesis or prosthetic implant which, in addition to providing stability by connecting the two adjacent vertebrae, allows for some relative movements therebetween, e.g. flexion and extension, and for damping when subjected to axial loads.

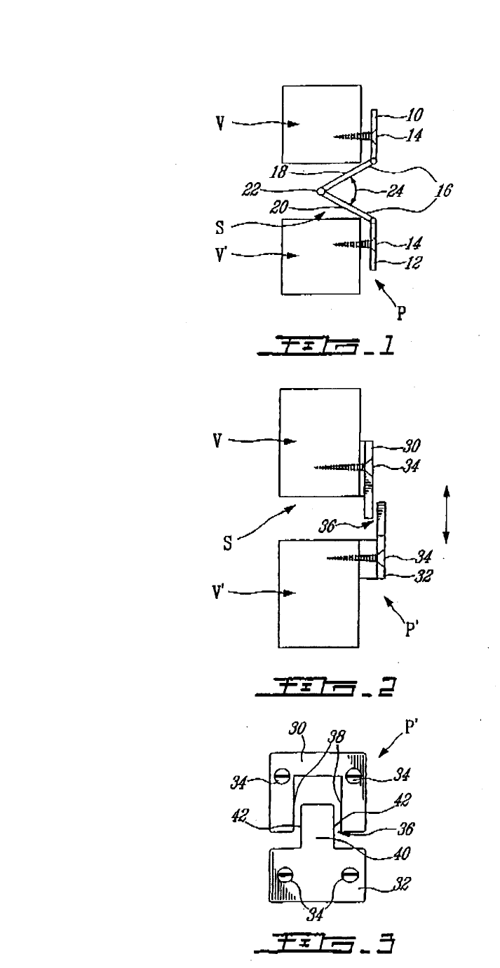

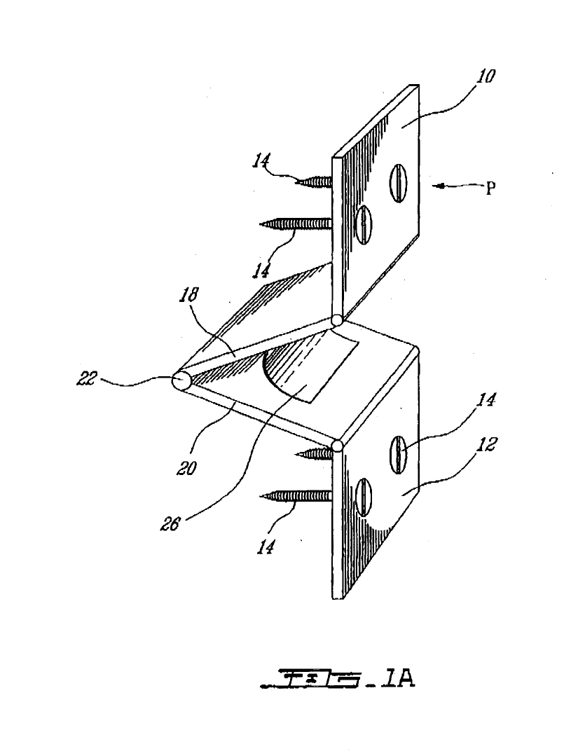

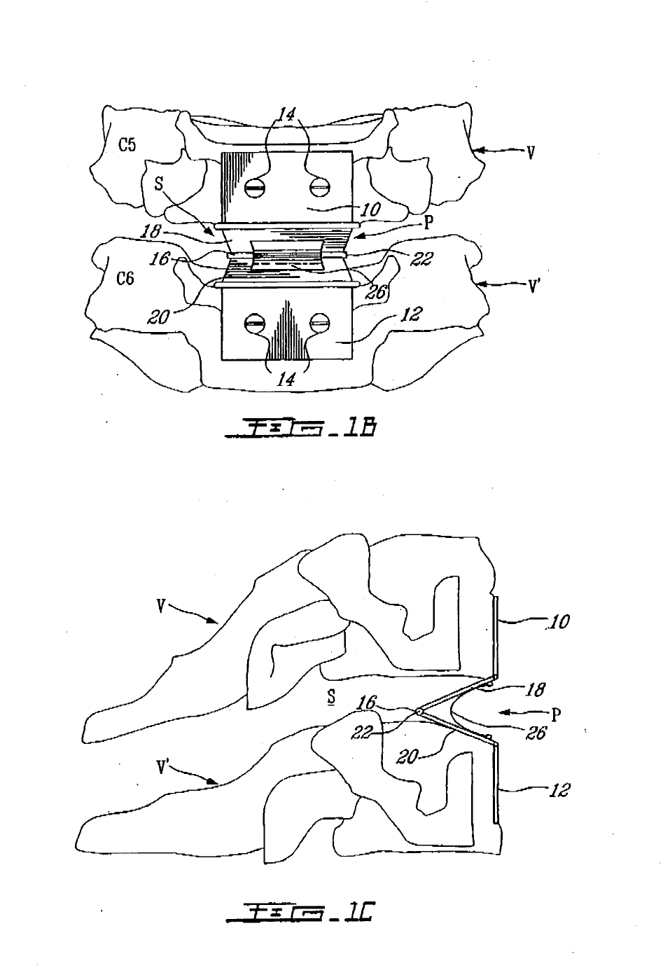

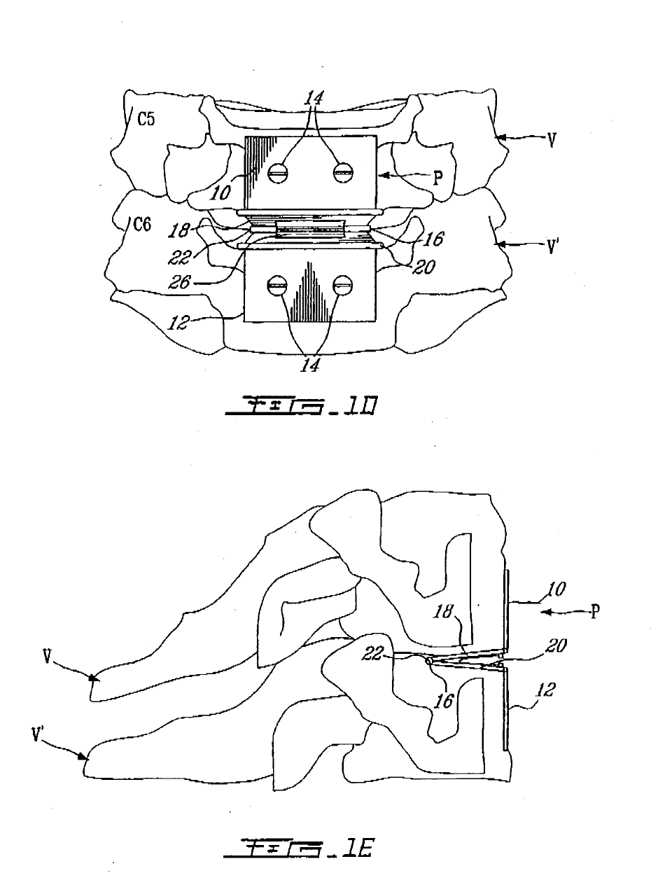

More particularly, the disc prosthesis P illustrated in FIGS. 1 and 1A comprises upper and lower anchoring plates 10 and 12, respectively, which are adapted to be secured with screws 14 to anterior faces of adjacent upper and lower vertebrae V and V′. The prosthesis P also includes a joint 16 connected to, and between, both plates 10 and 12 to link both vertebrae V and V′ in a stable manner and further providing damping characteristics to the prosthesis P and relative movements between the vertebrae V and V′. The joint 16 comprises a pair of upper and lower arms 18 and 20, respectively, which define a V-shaped configuration extending rearwardly from an anterior face of the vertebrae V and V′ and into the intervertebral space S defined vertically between the vertebrae V and V′. The upper and lower arms 18 and 20 can pivot such as with a hinge, at posterior ends thereof, i.e. at the apex 22 of the joint 16. The arms 18 and 20 of the joint 16 are biased, as per arrows 24 in FIG. 1, towards an open or expanded position thereof, for instance by way of a spring 26 (best shown in FIGS. 1A and 1C) in the form of a arcuately folded sheet, such that upon a movement of the rachis which brings the two vertebrae V and V′ closer together, the joint 16 closes against the spring force, the arms 18 and 20 pivoting towards each other about the apex 22, with the spring 26 being adapted to return the joint 16 to its at rest position once the effort made by the user that moves the rachis is released.

More than one spring may be used for maintaining, at rest, the joint 16 in an intermediate position, i.e. in a “floating” position such that the joint 16 is capable of opening or closing, with the spring forces always bringing it back to its at rest position. The upper and lower arms 18 and 20 of the joint 16 may be integral with the upper and lower plates 10 and 12, respectively.

The joint 16 substantially ensures three functions of the natural disc: stability by providing continuity between the adjacent vertebrae V and V′, damping in the axial plane: and flexion-extension movements in the sagittal plane. Depending on the material used for making the joint 16 (biocompatible or not), the joint 16 may be housed in a scaled chamber. The prosthesis P is, for instance, well adapted for the use on the cervical rachis.

In FIGS. 1B to 1E, the joint 16 is shown in extended and flexed positions thereof.

The joint 16 can also comprise one or more dynamometers; a system of one or more fluid-based dampers, i.e. with liquid(s) or gas(es); a bag to replace the natural disc's annulus, which is filled with a liquid, or other appropriate substance, having a proper viscosity index to replace the nucleus pulposus; etc.

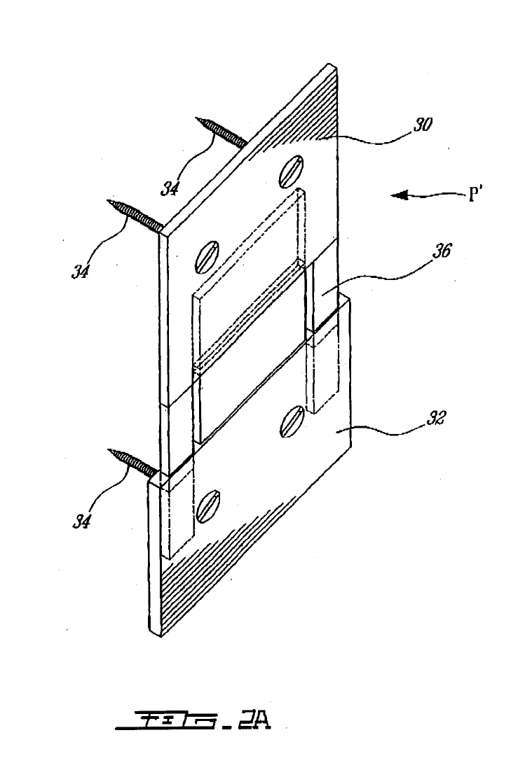

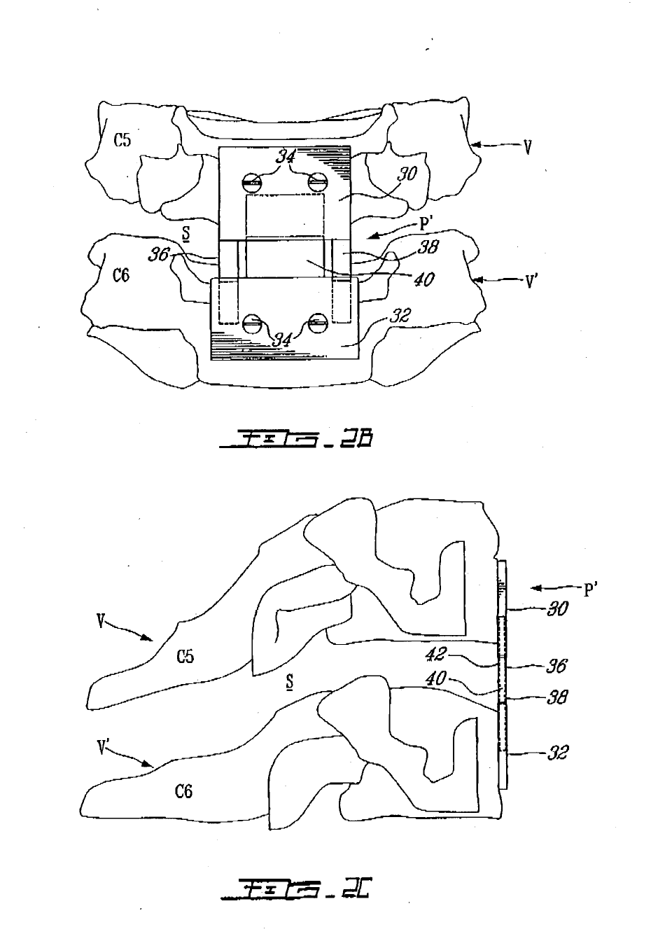

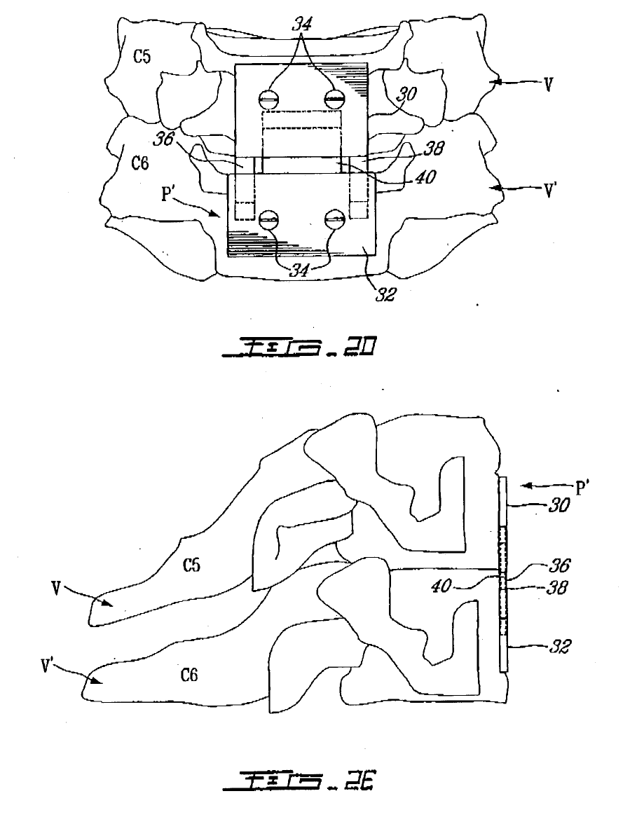

In FIGS. 2 and 3, there is shown a second embodiment of a disc prosthesis P′ also in accordance with the present invention and which has a cigar-cutter configuration, being located completely anteriorly of the upper and lower vertebrae V and V such as to provide for translational displacements along an axial plane between the vertebrae V and V (as opposed to the pivoting movement of the first disc prosthesis P of FIGS. 1 and 1A).

The second prosthesis P (see FIGS. 2, 2A and 3) comprises upper and lower anchoring plates 30 and 32, respectively, which are adapted to be secured with screws 34 to anterior faces of the adjacent upper and lower vertebrae V and V′. This second prosthesis P′ may also be used on the various vertebrae of the rachis, including advantageously on the cervical rachis. The prosthesis P′ also includes a joint 36 connecting both plates 30 and 32 to link both vertebrae V and V′ in a stable manner and further providing damping characteristics to the prosthesis P′ and relative movements between the vertebrae V and V′. The upper plate 30 may be inverted U-shaped and define side guide ways 38 while the lower plate 32 defines an extension 40 that is slidably engaged at its longitudinal sides 42 in the guide ways 38. A stop member (not shown) is provided for preventing the complete withdrawal of the lower plate 32 from the upper plate 30 in the event of hyperextension by the patient.

As in a cigar cutter, a spring (not shown) is preferably provided between the upper and lower plates 30 and 32, for instance in the guide ways 38, such that the prosthesis P is biased towards its extended position.

Alternatively, the spring effect may be provided in the intervertebral space S defined between the vertebrae V and V′, i.e. posteriorly of the plates 30 and 32, such a by a coil spring extending vertically between, and linking, both vertebrae V and V′, or by a damping unit consisting for example of a bag containing a fluid (liquid or gaseous). Also, the plates 30 and 32 could include a substantially horizontal posterior intersomatic extension, located in the space S and between which a bias system, e.g. a spring or fluid damper, would be provided.

For the cervical rachis, the plates 30 and 32 are concave to respect the natural cervical lordosis of the anterior wall of the cervical spine and to guide harmonious flexion-extension movements.

In FIGS. 2B to 2E, the joint 36 is shown in extended and flexed positions thereof.

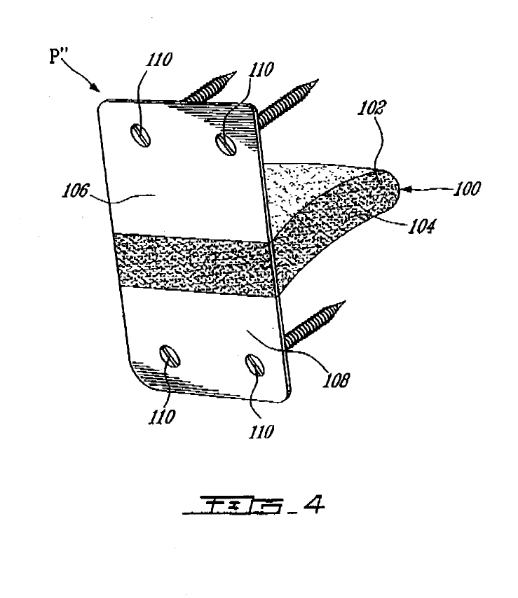

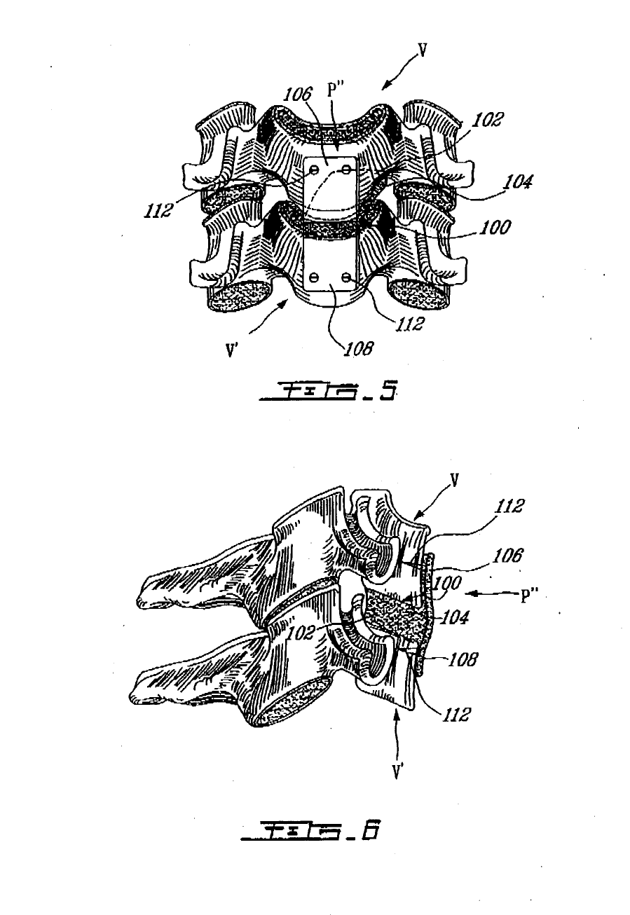

In FIGS. 4 to 6 which show a third embodiment also in accordance with the present invention, a further disc prosthesis P″ is illustrated and is, in fact, a one-piece tissue jacket 100 defining a posterior bi-concave constraining chamber 102 adapted to receive therein a hydrogel 104 that acts as a damper, with anterior frontally extending upper and lower extensions 106 and 108, respectively, adapted to be anchored to the anterior faces of the facing upper and lower vertebrae V and V′ with screws 112 that extend through reinforced eyelets 110.

More particularly, the bi-concave hydrogel 104 of the joint of disc prosthesis P″ conforms to or mimics the natural shape of a cervical disc (16, 18 or 20 mm depth×6, 8 or 10 mm height) and is surrounded or coated with the deformable constraining jacket 100 located in an intra-spinal inter-somatic space. The pair of frontal, extra-spinal and pre-somatic, upper and lower extensions 106 and 108 extend respectively from the antero-superior and antero-inferior edges of the jacket 100.

As to the nucleus core 104 of this third disc prosthesis P″, it is made of a hydrogel, which is non-biodegradable and is chemically reticulated by covalent bonds, and which has visco-elastic properties that are similar to those of the natural nucleus pulposus such as to counterbalance or offset the external hydrostatic pressure which is exerted thereon. The hydrogel has a swelling or inflating capability in an aqueous solution of about 85 to 95%, at equilibrium (WG). The hydrogel can be a terpolymer formed of: (a) a methacrylamide N-substitute, for instance [N-2 (hydroxypropyl methacrylamide)](HPMA); (b) a hydroxy alkyl methacrylate ester, for instance 2-hydroxyethyl methacrylate (HEMA); and (c) a di- or tri-ethylene glycol dimethacrylate (DEGDMA or TEGDMA).

While it is manufactured, the hydrogel 104 is dehydrated and inserted in the intervertebral cavity. Then, it is manually rehydrated in an aqueous solution by using a needle puncture through the coating jacket 100 until its maximal swelling capability (WC). The hydrogel is prepared in such a way that WG corresponds to a pre-selected specific volume of the intervertebral bi-concave chamber 102 in order to obtain the adequate pressure. This hydrogel forming the nucleus core, should as much as possible have the deformation properties and the coherence characteristics of the natural nucleus pulposus in order to respectively have dampening curves compatible with the typical levels of mechanical loads of natural lumbar discs and have resistance to fracturing under applied pressures.

The tissue jacket 100 should have an intrinsic resiliency, or memory, that gives it a tendency to keep its bi-concave nucleus-like shape during its displacement in the cavity and so maintain contact with the natural vertebra endplates. It should also have enough compliance such as not to modify motions and dampening properties of the hydrogel-nucleus.

Alternatively, the nucleus hydrogel 102 may be shaped in a series of independent flexible micro-beads (e.g. spheres containing appropriate fluid for damping effect).

Finally, the above three (3) prostheses P, P′ and P″, which are adapted to be installed by an anterior approach on any of the cervical, lumbar, dorsal and thoracic rachis, could also be of a multi-level configuration, that is to cover more than two adjacent vertebrae. The holes defined in the anchoring plates 10/12 and 30/32 or anchoring extensions 106/108 may be vertically elongated (oblong) to allow for some adjustment in the positioning of the prosthesis P/P′/P″ and so that the latter may be used with patients of various vertebra configurations and sizes.

Therefore, the prosthesis of the present invention constitutes a system that attaches two vertebrae together while allowing for relative movements, e.g. pivoting, translational or other, between these vertebrae and while providing some spring force or damping therebetween.

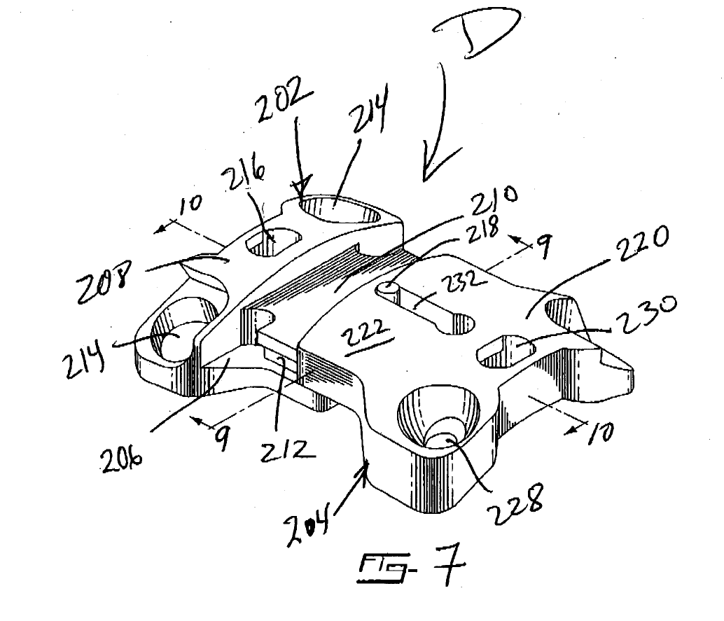

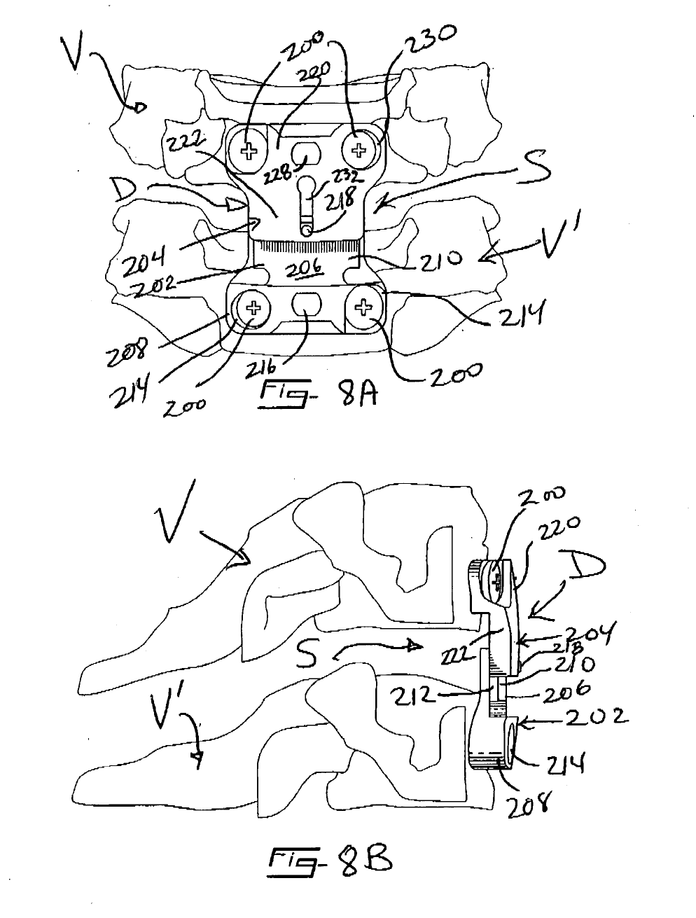

FIGS. 7 to 10 illustrate a device D, also in accordance with the present invention, which is used to connect two adjacent vertebrae V and V′. The device D is typically used when a natural disc, or part thereof, is removed from between the two adjacent vertebrae V and V′. The disc is replaced, for instance, by a bone graft (not shown in the drawings) that is positioned in the intervertebral, or intersomatic, space S defined between the two vertebrae V and V′. The device D is secured to both vertebrae V and V′ via screws 200 that extend though the device D and into both vertebrae V and V′ (see FIGS. 8A and 8B). As the bone graft takes months to fuse with these two vertebrae V and V′and as it reduces in size (graft subsidence according to Wolff′s laws) while doing so (i.e. the bone graft contracts, e.g. from 1 to 2 mm for a bone graft having an initial height of 8 to 10 mm), any rigid assembly connecting the two vertebrae will impart, as mentioned hereinbefore, immense stresses (shield effect) generally to itself, rather than to the vertebrae V and V′ and the bone graft.

Therefore, in order to overcome this shortcoming, the present device D is not of rigid unitary construction, but is rather translationally extendable for accommodating the slow and gradual relative displacement between the vertebrae V and V′ that takes place during the integration of the bone graft thereto. Indeed, as the bone graft subsides and fuses to the vertebrae V and V′, it contracts thereby reducing the height of the intervertebral space S, whereby the vertebrae V and V′ are drawn closer together. To accommodate this movement of the vertebrae V and V′ and substantially eliminate the above-mentioned stresses, the present device D itself vertically contracts; in other words, the device D follows the relative movement of the vertebrae V and V′,without stress being induced to the device D, the screws 200, the vertebrae V and V′ or the bone graft To do so, the device D, which is the form of a plate, comprises upper and lower plate members 202 and 204 that are adapted to be secured, via the screws 200, respectively to the upper and lower vertebrae V and V′. The upper and lower plate members 202 and 204 arc slidable engaged one to the other such as to be able to relatively displace in the direction of the movement of the vertebrae V and V′.

The device D could be provided with an anti-reverse mechanism to allow the upper and lower plate members 202 and 204 only to move towards each other, that is in the direction of subsidence (or during flexion movements), while preventing the device D from elongation (i.e. preventing the upper and lower plate members 202 and 204 from becoming increasingly spaced) during extension movements. Such an anti-reverse mechanism would maintain the bone graft permanently in a compressed state such as to favour its fusion (Wolff's laws) and prevent its gaping during extension movements of the neck. This mechanism could take the form of a pair of slides having cooperating ratchet-like teeth that permit a relative displacement of the upper and lower plate members 202 and 204 towards each other. A material having a memory could also be used. The device D thus allows for translational semi-constrained flexibility.

More particularly, the upper plate member 202 includes a base 206 and a head 208. the base 206 includes a T-shaped male guide element 210 that defines a pair of parallel longitudinal side guide ways 212. The head 208 defines a pair of lateral openings 214 through which the screws 200 are passed, and a central aperture 216 adapted to temporarily receive, in a set position, an alignment mechanism (e.g. localizer) during installation of the screws 200 through the device D and into the vertebrae V and V′. A pin 218 extends perpendicularly from the base 206.

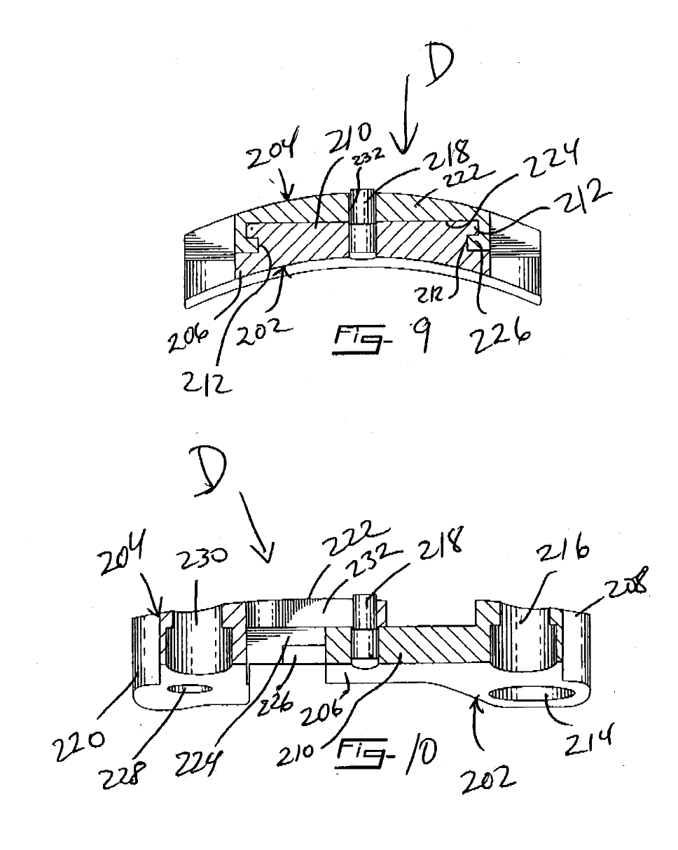

The lower plate member 204 includes a head 220 and a female guide element 222. The female guide element 222, as best seen in FIG. 9, comprises a broad channel 224 that defines a pair of parallel longitudinal side guides 226. The female guide element 222 and its channel 224 and guides 226 receive in a sliding relationship the male guide element 210 and guide ways 212, so that the upper and lower plate members 202 and 204 can translationally displace relative to another. The head 220 defines a pair of lateral openings 230 through which the screws 200 are passed, and a central aperture 228 used as aperture 216 of the upper plate member 202. The female guide element 222 also defines a longitudinal slot 232 through which extends the pin 218 of the base 206 of the upper plate member 202, so as to further guide the upper and lower plate members 202 and 204 in their longitudinal relative displacements.

The device D is curved at its anterior and posterior faces to respect adjacent tissues (bone posteriorly and oesophagus anteriorly) while its sliding internal mechanism could be linear or curved.

To connect more than two consecutive vertebrae, the device can have longitudinal ends that are of half-height so that two or more devices can be arranged in an end-to-end relationship with the longitudinal ends of two adjacent devices overlapping and with the screws being passed through these overlapping ends.

The device D can be used for all types of vertebrae, including cervical, dorsal, lumbar and sacral vertebrae.

It is noted that, in FIGS. 8A and 8B, the device D is shown in inverted position, wherein the upper and lower plate members 202 and 204 are secured respectively to the lower and upper vertebrae V′ and V. The device D can assume both orientations.

The device D could also be used instead of the sliding plate P′ in the arrangement of FIGS. 2A to 2E, i.e. wherein the plate is used in combination with a damping system in lieu of the rigid bone graft of the embodiment of FIGS. 7 to 10.

1. A connecting device for use in attaching together at least two adjacent vertebrae, comprising first and second plate members adapted to be fixedly secured respectively to upper and lower vertebrae of a pair of adjacent vertebrae, said first and second plate members being provided with respective first and second mating members, said first and second mating members being engaged so as to allow a relative displacement between said first and second plate members in response to a similar relative displacement between the upper and lower vertebrae that alters a spacing therebetween.

2. A device as defined in claim 1, wherein there is provided an anti-reverse mechanism adapted to allow said first and second plate members only to move relatively towards each other while preventing said first and second plate members from becoming increasingly spaced.

3. A device as defined in claim 2, wherein said anti-reverse mechanism comprise first and second members, slidably engaged together and provided with cooperating ratchet-like teeth adapted to permit only a relative displacement of said first and second plate members towards each other.

4. A device as defined in claim 2, wherein said anti-reverse mechanism comprise a material having a memory adapted to permit only a relative displacement of said first and second plate members towards each other.

5. A device as defined in claim 1, wherein said first and second mating members comprise male and female guide elements slidably engaged together for allowing a translational movement between said first and second plate members.

6. A device as defined in claim 5, wherein said male guide element comprises longitudinal guide ways, and wherein said female guide element comprises a channel for slidably receiving said male guide element and a pair of side guides extending inwardly and slidably engaged in said guide ways of said male guide element.

7. A device as defined in claim 6, wherein said female guide element defines a longitudinal slot, and wherein said male guide element comprises a pin extending perpendicularly to a direction of said relative displacement and engaged in said slot.

8. A method of connection of at least two adjacent vertebrae, comprising the steps of:
a) removing at least part of a disc from an intervertebral space defined between a pair of adjacent vertebrae that include upper and lower vertebrae;
b) securing first and second plate members respectively to the upper and lower vertebrae, said first and second plate members being capable of relatively displacing for accommodating a change in a distance between the upper and lower vertebrae;
wherein after step a), a bone graft is positioned in the intervertebral space.
